(19) United States
(12) Patent Application Publication
(10) Pub. No.: US 2005/0262617 A1
(43) Pub. Date: Dec. 1, 2005

(54) Absorbent cranial cap

(76) Inventor: Andrew Szypka, Curtice, OH (US)

Correspondence Address: PURDUE LAW OFFICES;2735 N. HOLLAND-SYLVANIA ROAD, SUITE B-2, TOLDEO, OH, 43615, US

(21) Appl. No.: 11/141,510

(22) Filed: May 31, 2005

Related U.S. Application Data
(60) Provisional application No. 60/575,654, filed on May 28, 2004.

(51) Int. Cl. A61F 2/30

(52) U.S. Cl. 2/410

(57) ABSTRACT

An absorbent cranial cap is disclosed. The cap comprises a lower liquid impervious backing layer and an upper, absorbent layer including superabsorbent material in an amount sufficient to absorb at least 100 ml of a 0.9% saline solution. The cap is useful in the funeral industry.

### BACKGROUND OF THE INVENTION

(1) Field of the Invention

The present invention relates generally to articles of manufacture that are useful in the funeral industry and, specifically, to absorbent articles that are useful in supporting a decedent's head during a showing.

(2) Description of Related Art Including Information Disclosed Under 37 CFR 1.97 and 37 CFR 1.98

A remarkable number of decedents are shown in open caskets during visitation. Sadly, in some cases, cranial leakage must be contended with during this delicate occasion. Cranial leakage can be caused by injuries or traumas sustained before death as well as by post mortem procedures carried out, for example, during an autopsy. In any case, if a corpse with a head subject to leakage is to be shown, the head must be supported, typically on a pillow, and leakage must be contained.

The state of the art product for containing cranial leakage is a cranial cap formed from cellulosic fibers into a three dimensional product that is generally trough shaped. The cranial cap is typically hidden between the head and a pillow in a casket. This cranial cap is treated to be moisture repellant so that head leakage is “caught” in the bottom of the trough.

In a search directed to the present invention, the following U.S. Pat. Nos. were noted: 6,440,111; 6,206,865; 5,444,900; 5,360,504; 5,091,240; 4,534,769; 3,881,490; 3,809,077; 3,768,127; 3,674,613; 2,730,788; and 2,709,293.

U.S. Pat. No. 5,091,240 discloses an absorbent article comprising a first layer of absorbent fibers bonded to a layer of closed cell foam. The fibers are bonded together with a water based adhesive.

U.S. Pat. No. 5,444,900 discloses a casket mattress capable of absorbing up to fifteen gallons of liquid. This patent discloses the use of super absorbents in an absorbent layer of the mattress.

U.S. Pat. No. 4,534,769 discloses a diaper including a foam backing sheet.

U.S. Pat. No. 3,881,490 discloses a feminine pad including a foam backing sheet (FIG. 4).

### BRIEF SUMMARY OF THE INVENTION

The present invention is an absorbent cranial cap including a lower liquid impervious backing layer and an upper, absorbent layer including superabsorbent material in an amount sufficient to absorb at least 100 ml of a 0.9% saline solution.

It is an object of the present invention to provide a highly absorbent cranial cap.

It is another object of the present invention to provide an absorbent cranial cap with a liquid impervious backing layer.

It is still a further object of the invention to provide a compact, thin cranial cap that outperforms the prior art cranial cap in terms of absorbent capacity and in terms of its ability to prevent pillow staining.

It is yet another object of the invention to provide a cranial cap which easily conforms to the shape of any decedent's head.

It is still another object of the invention to provide a cranial cap that is large enough for the biggest head and can be trimmed to fit any size head.

It is a still further object of the invention to provide a cranial cap that can perform all of the functions required of it yet remain fully hidden from view and be not visible to viewers.

These and other objects and advantages of the present invention will be fully appreciated by those skilled in the art upon reviewing the disclosures herein.

### BRIEF DESCRIPTION OF THE SEVERAL VIEWS OF THE DRAWING

FIG. 1 is a plan view of a cranial cap according to the present invention.

FIG. 2 is a view, in cross section, taken along the line 2-2 of FIG. 1.

FIG. 3 is a side, cross-sectional view of a cranial cap according to the invention positioned for use between a pillow and the head of a decedent.

### DETAILED DESCRIPTION OF THE PREFERRED EMBODIMENTS

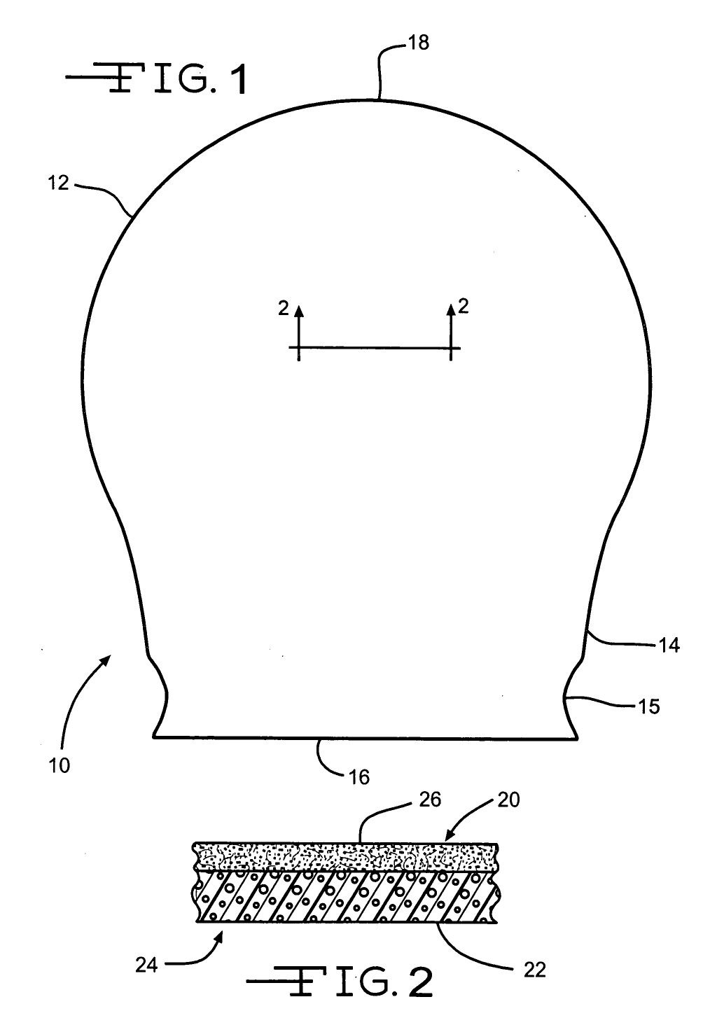

Referring to FIG. 1, a cranial cap according to the present invention is indicated generally at 10 and, in a preferred embodiment, comprises a head portion 12 and a neck portion 14. In use, the neck portion 14 can be tucked into a decedent's collar to properly position and align the cap 10. The neck portion 14 has two cut-outs indicated at 15 and terminates in a first end 16 which preferably has a width of about 200 millimeters (“mm”) or less and more preferably, a width of about 185 mm. From the first end 16 to a top end 18, the cap 10 preferably has a length of about 250 mm. At its widest point, the cap 10 preferably has a width of about 250 mm. The head portion 12 of the cap 10 is generally round and the neck portion 14 is generally rectangular or trapezoidal, except for the cut-outs 15. Preferably, the neck portion 14 has a width that is less than the diameter of the head portion 12.

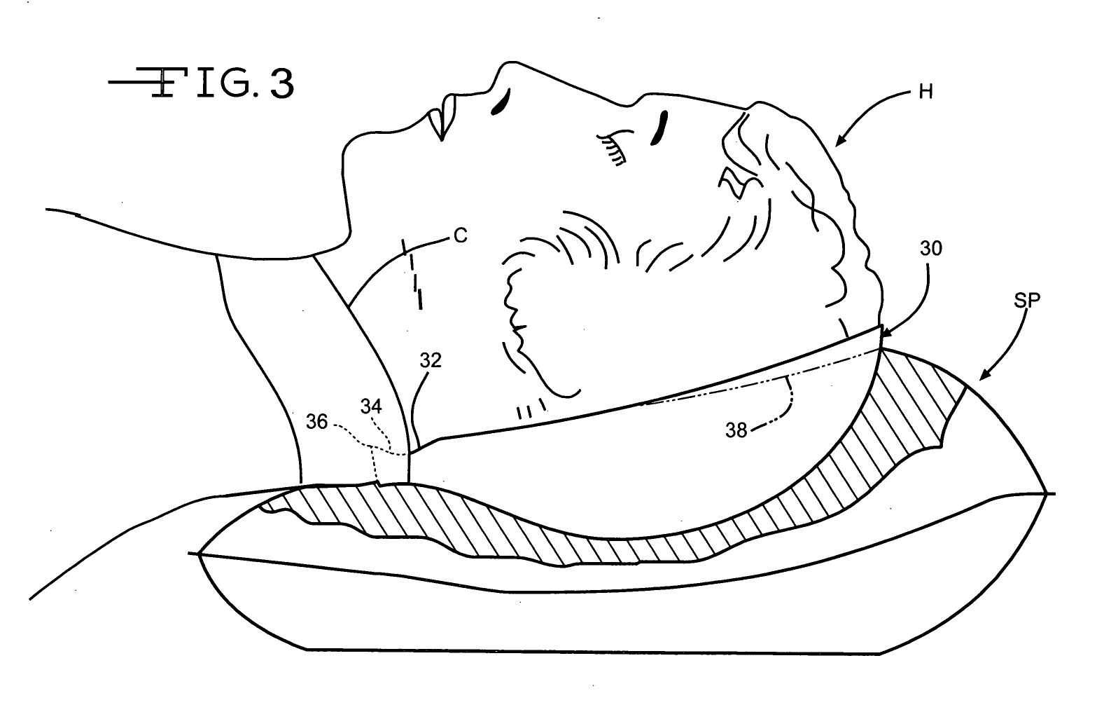

Referring now to FIG. 2, the cap 10 comprises a first layer 20 and a second layer 22. The first, upper layer 20 is an absorbent layer that is bonded to the second layer 22, which, preferably, is substantially moisture impervious. For the upper layer 20, a preferred material is an airlay web formed of cellulosic fibers, a thermal binder fiber and superabsorbent fibers. The airlay web has a tissue layer on one side and that is the exposed side which will contact the head of a decedent. A suitable and preferred material is available commercially from Bandz, Inc. under the trade name ThermaCore under the designation 280/40. This material has a nominal thickness of about three thirty-seconds of an inch and a basis weight of 280 grams per square meter. Forty percent by weight of the material is constituted by superabsorbent fibers, which are polyacrylate superabsorbent fibers. This material provides approximately about 5.5 grams of superabsorbent fibers in a cap having the preferred dimensions recited above and the cap can absorb and hold 300 ml of 0.9% saline solution. This absorbed liquid will not, for the most part, squeeze out under pressure because superabsorbent materials lock liquid in and prevent it from being squeezed out or from migrating. Thicker material or material having a higher concentration of superabsorbent fibers may be employed if increased absorbency is desired. Superabsorbent particles may be incorporated in the upper layer 20 in lieu of superabsorbent fibers. In any case, a cap according to the present invention includes a superabsorbent material in an amount sufficient to absorb and hold at least 100 ml of 0.9% saline solution. Preferably, a cap according to the present invention includes a superabsorbent material in an amount sufficient to absorb and hold at least 150 ml of 0.9% saline solution and, most preferably, includes a superabsorbent material in an amount sufficient to absorb and hold at least 200 ml of 0.9% saline solution.

For the lower layer 22, a closed cell polyethylene foam material having a nominal thickness of one eighth of an inch is preferred. The first and second layers 20 and 22 are bonded together to form a composite laminate indicated generally at 24. Any suitable bonding method may be used although presently a hot-melt adhesive bonding method is preferred where hot melt adhesive is applied to an upper face 26 of the lower layer 22 and the upper layer 20 is pressed onto and into the hot melt adhesive and the two layers 20 and 22 are bonded under pressure.

The composite laminate 26 can be formed in-line with stock material and individual caps 10 can be die-cut from the composite laminate sheet. The shape of the cap 10 minimizes waste in such a die-cutting operation.

The use of polyethylene foam for the lower layer 22 offers numerous advantages, especially in comparison to the formed cellulosic prior art cranial cap. The prior art cap is three dimensional and bulky while the cranial cap 10 of the present invention is flat, thereby minimizing precious packaging required for shipping and storage of the cap 10. The composite laminate 26 used in cranial caps according to the present invention are fully conformable to the individual shape of any decedent's head while the stiff prior art cap is not readily conformable. The prior art cap will easily slide on a satin pillow of the type commonly used by funeral directors while the foam layer 22 will not readily slide thereon, thereby facilitating the task of preparing a decedent for a showing. Further, the prior art cranial cap is merely moisture resistant while the cranial cap according to the present invention includes a lower layer 22, such as closed cell polyethylene foam which is substantially liquid impervious so that staining of a satin pillow, for example, is proactively prevented.

Referring now to FIG. 3, a cranial cap 30 is shown in use between a satin pillow SP and the head of a decedent H. An upper portion of the pillow P is shown in cross section to illustrate how the cranial cap 30 conforms, as required, to the decedent's head. The cranial cap 30 includes a neck portion 32 that is conveniently tucked into a collar C on the decedent's clothing. The neck portion 32 includes two cut-outs, one of which is indicated at 34, and the cut-outs are positioned within the collar C of the decedent's clothing. Adjacent to the cut-out indicated at 34, there is a collar gripping point 36 which will help locate the cap 30 relative to the collar and will frictionally retain the cap 30 in the selected location. A dotted line 38 shows where one could trim the cap 30 to so that it would be small enough to be hidden completely in use. The cap 30, as shown, is too large because it extends beyond the area of contact between the satin pillow SP and the decedent's head H. If the pad is trimmed along the dotted line 38 and replaced under the decedent's head H, the cap 30 will be completely hidden from view in use and this is preferred.

It will be appreciated that the foregoing detailed description is made to comply with the requirement that applicants disclose the best mode known to them for practicing the invention. It will also be appreciated, however, that the invention described above is susceptible of modifications and changes without departing from the spirit and scope of the invention as defined in the appended claims.

1. A cranial cap for preventing liquid exuded from a decedent's cranial area from staining a pillow placed thereunder, said cranial cap comprising
a first, upper absorbent layer comprising superabsorbent material in a quantity sufficient to absorb at least 100 ml of 0.9% saline solution,
a second, lower, substantially liquid impervious layer bonded to said first layer to produce a composite laminate that conforms to the shape of a decedent's head when placed between a decedent's head and a pillow.

2. A cranial cap for preventing liquid exuded from a decedent's cranial area from staining a pillow placed thereunder, said cranial cap being generally flat when not in use and comprising
a head portion,
a neck portion, wherein at least said head portion comprises
a first, upper absorbent layer having first and second major surfaces, said first layer comprising cellulosic material and superabsorbent material, said first layer including a quantity of superabsorbent material sufficient to absorb at least 100 ml of 0.9% saline solution,
a second, lower, substantially liquid impervious layer having first and second major surfaces, said first major surface of said second layer being bonded to said first major surface of said first layer to produce a composite laminate that conforms to the shape of a decedent's head when placed between a decedent's head and a pillow.

3. The cap claimed in claim 2 wherein said first layer includes a tissue layer and it constitutes the second major surface of said first layer

4. The cap claimed in claim 1 wherein said first layer includes a quantity of superabsorbent material sufficient to absorb at least 150 ml of 0.9% saline solution.

5. The cap claimed in claim 2 wherein said first layer includes a quantity of superabsorbent material sufficient to absorb at least 150 ml of 0.9% saline solution.

6. The cap claimed in claim 1 wherein said first layer includes a quantity of superabsorbent material sufficient to absorb at least 200 ml of 0.9% saline solution.

7. The cap claimed in claim 2 wherein said first layer includes a quantity of superabsorbent material sufficient to absorb at least 200 ml of 0.9% saline solution.

8. The cap claimed in claim 1 wherein said second layer is comprised of closed cell foam.

9. The cap claimed in claim 2 wherein said second layer is comprised of closed cell foam.

10. The cap claimed in claim 3 wherein said second layer is comprised of closed cell foam.

11. The cap claimed in claim 4 wherein said second layer is comprised of closed cell foam.

12. The cap claimed in claim 5 wherein said second layer is comprised of closed cell foam.

13. The cap claimed in claim 6 wherein said second layer is comprised of closed cell foam.

14. The cap claimed in claim 7 wherein said second layer is comprised of closed cell foam.
